(19) United States
(12) Patent Application Publication
(10) Pub. No.: US 2026/0325800 A1
(43) Pub. Date: Oct. 22, 2026

(54) Syringe for Single-Handed Guidewire Manipulation and Syringe Support

(71) Applicant: Centripetal Perfusion LLC, Madison, WI (US)

(72) Inventors: Josh Glazer, Madison, WI (US); Adam McNeilly, LaCrosse, WI (US)

(21) Appl. No.: 19/119,306

(22) PCT Filed: Oct. 31, 2023
(86) PCT No.: PCT/US2023/036435

§ 371 Date: Apr. 8, 2025

Related U.S. Application Data
(60) Provisional application No. 63/422,221, filed on Nov. 3, 2022.

(51) Int. Cl. A61M 25/09

(52) U.S. Cl. CPC A61M 25/09041

(57) ABSTRACT

A syringe for use in inserting guidewires, for example, using the Seidinger technique, provides a bore through the piston of the syringe plunger preceded by a guidewire support surface on the syringe plunger handle. The guidewire support surface may be exposed to allow the guidewire to be retracted or extended by pressure of the user's finger on the guidewire against the guidewire support surface.

### CROSS REFERENCE TO RELATED APPLICATION

This application claims the benefit of U.S. provisional application 63/422,221 filed Nov. 3, 2022 and hereby incorporated by reference.

### BACKGROUND OF THE INVENTION

The present invention relates generally to syringes used during the insertion of a guidewire into a blood vessel and in particular to a syringe providing improved stabilization and manipulation of the syringe assembly and guidewire using one hand.

The preferred technique for accessing intravascular and other internal anatomic targets during medical procedures relies heavily on the classic Seldinger technique whereby a needle is introduced into the target location and then a separate wire is inserted via the needle.

This technique can be challenging to perform because the needle tip can become dislodged from the target and/or injure adjacent tissues as the proceduralist removes the syringe and introduces the wire into the needle. Additionally, because the modern standard of care requires use of ultrasound guidance during these procedures, the classic two-handed, multi-step Seldinger technique cannot be performed with complete ultrasonographic guidance throughout some of the most crucial steps.

### SUMMARY OF THE INVENTION

The present invention provides a syringe having a guidewire support surface integrated into the handle of the syringe plunger, the latter of which also includes a bore providing passage of the guidewire along the support surface and through the plunger into the needle. The guidewire may then be moved by physician's finger pressing the guidewire against the support surface while the physician stabilizes the syringe body with other fingers of the same hand. The syringe thus allows single-handed operation freeing the physician's other hand for use, for example, to support an ultrasound probe or the like.

In one embodiment, the invention provides a syringe assembly having a syringe tube with a needle connector at a first end in fluid communication with a volume of the syringe tube. The syringe assembly also includes a syringe plunger providing a piston slidably received within the syringe tube and a handle portion extending from a second end of the syringe tube. The syringe plunger provides a bore passing through the piston to allow the passage of a guidewire received within the second end of the syringe tube to extend through the piston to the needle connector. The handle portion presents a guidewire support surface against which the guidewire may be pressed to advance or retract the guidewire through the bore.

It is thus a feature of at least one embodiment of the invention to provide a surface on the plunger handle, close to a user's hand when that hand is used to support the syringe tube, that can be used to advance or retract the guidewire, for example, with a single finger of the same hand supporting the syringe tube.

The guidewire surface may be exposable with movement of the syringe plunger outside of the syringe tube so that the guidewire may be pressed by a finger of the user's hand supporting the syringe tube.

It is thus a feature of at least one embodiment of the invention to provide an extremely simple design allowing direct contact between the user's finger and the guidewire when the syringe tube is supported by the same hand.

The guidewire support surface may include ridges extending away from the handle and across a length of the guidewire when the latter is positioned within the syringe assembly.

It is thus a feature of at least one embodiment of the invention to isolate contact between the user's finger and the handle to concentrate pressure on the guidewire.

The syringe assembly may further include a guidewire holder attached to the handle portion for dispensing a guidewire from guidewire holder to the syringe handle.

It is thus a feature of at least one object of the invention to eliminate the need for the user to separately support a guidewire holder, for example, using a second hand.

The guidewire holder may attach to an end of the handle portion removed from the piston and may provide a slot extending from the guidewire holder to the guidewire support system and open to allow insertion of a guidewire therethrough in a direction perpendicular to a direction of advancing or retracting the guidewire through the bore.

It is thus a feature of at least one embodiment of the invention to simplify installation of the guidewire into the plunger.

The needle connector may provide a central lumen along an axis and the bore may be aligned with the axis. In addition, movement of the syringe plunger may be along the axis.

It is thus a feature of at least one embodiment of the invention to provide a direct path of the guidewire without unnecessary twisting or bending of the guidewire.

The syringe assembly may further include a guide tube extending from the needle connector toward the syringe plunger.

It is thus a feature of at least one embodiment of the invention to reliably guide the guidewire from the piston to the needle connector when the piston is displaced from the needle connector, for example, after aspirating some blood into the syringe volume.

The syringe plunger may provide an axial bore and wherein the guide tube may be sized to be received within the axial bore for at least some positions of the syringe plunger within the syringe tube.

It is thus a feature of at least one embodiment of the invention to prevent the guidewire from being obstructed or caught by the relatively blunt end of the syringe tube outside of the needle connector.

The syringe assembly may further include a seal positioned within the bore to restrict the passage of liquids through the bore while the guidewire is in place in the seal.

Thus it is a feature of at least one embodiment of the invention to allow normal use of the syringe for aspiration while the guidewire is in place, reducing the need for additional manipulation of the guidewire during the procedure.

These particular objects and advantages may apply to only some embodiments falling within the claims and thus do not define the scope of the invention.

### BRIEF DESCRIPTION OF THE DRAWINGS

FIG. 1 is a perspective view in partial fragment and phantom of a syringe assembly of the present invention showing an attached guidewire holder communicating with the handle of the syringe plunger (rotated 90° for clarity) to feed a guidewire across a guidewire support surface through the plunger piston and out of the needle connector;

FIG. 2 is an elevational cross-section of the syringe tube and plunger of FIG. 1 showing the guidewire support surface in more detail and also showing an internal seal within the bore with the guidewire holder in exploded arrangement;

FIG. 3 is a fragmentary perspective view showing one possible positioning of a user's hand for manipulating the guidewire;

FIG. 4 is an elevational cross-section through the guidewire support surface showing ribs increasing the pressure of the user's finger against the guidewire versus against the handle;

FIG. 5 is a side elevational view of an alternative embodiment of the guidewire support surface showing a living hinge pawl for advancing the guidewire across the support surface with flexure of the pawl by the user's finger; and

FIG. 6 is a cross-sectional elevational view of an alternative embodiment of the syringe plunger including an insert into which the guidewire may be pre-threaded and which may attach to the guidewire holder.

### DETAILED DESCRIPTION OF THE PREFERRED EMBODIMENT

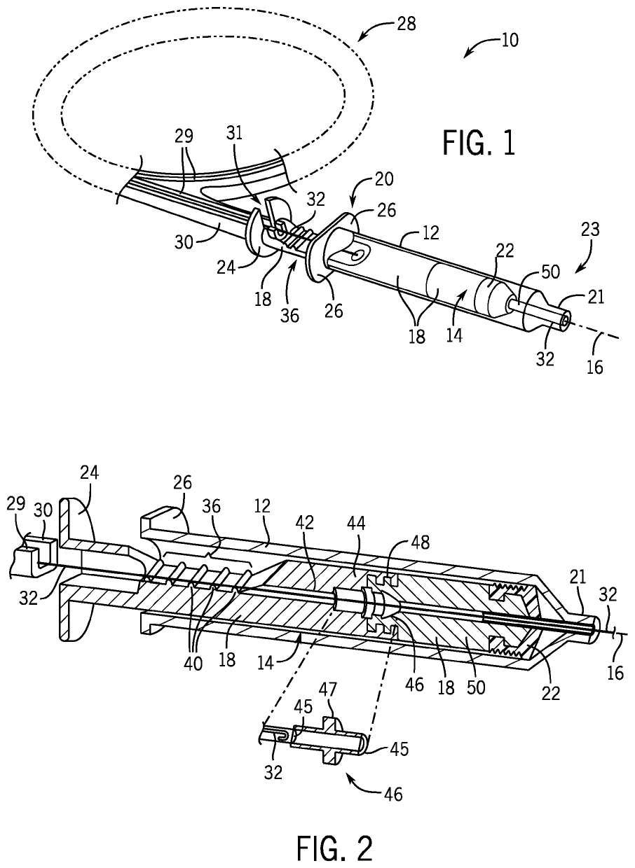

Referring now to FIG. 1, a syringe assembly 10 according to one embodiment of the present invention provides a generally cylindrical syringe tube 12 open at a proximal end 20 to receive a plunger 14 fitting coaxially therein. The plunger 14 may move within the syringe tube 12 along an axis 16 under the influence of force applied to a handle 18 of the plunger 14 extending out of a proximal end 20 of the syringe tube 12.

Within the syringe tube 12, the distal end of the handle 18 of the plunger 14 may attach to an elastomeric piston 22 having an outer circular periphery providing a sliding seal against inner walls of the syringe tube 12. The distal end 23 of the syringe tube 12 necks inwardly to a needle connector 21, for example, providing a Luer lock or slip-tip connection (as shown) suitable for liquid-tight connections to standard hypodermic needles and the like.

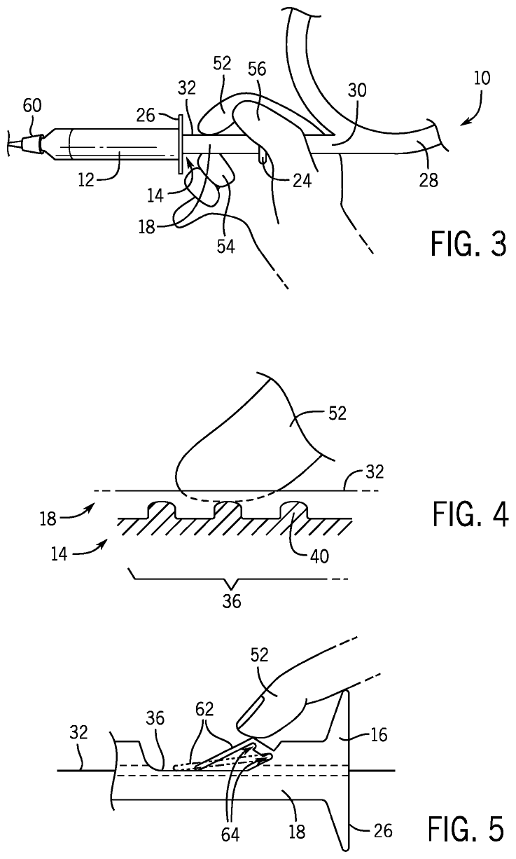

A proximal end of the handle 18 provides a radially outwardly extending flange 24 offering purchase for a user's finger when inserting or withdrawing the plunger 14 along the axis 16 with respect to the syringe tube 12, a process further assisted by outwardly extending finger stops 26 attached at the proximal end of the syringe tube 12 as will be discussed below with respect to FIG. 3.

The handle 18, syringe tube 12, and elastomeric piston 22 may, for example, be constructed of a thermoplastic polymer so as to be injection molded.

The proximal end of the handle 18 may attach to a guidewire dispenser 28, the latter, in one embodiment, providing generally a helical trough 29 into which a metal guidewire 32 may be coiled and extracted at a tangential exit trough 30. The tangential exit trough 30 provides a point of attachment to the handle 18, and in one embodiment, the trough 29 may continue as a slot 31 through the flange 24 allowing the guidewire 32 to be readily inserted into the syringe tube 12 along the axis 16.

As so inserted, the guidewire 32 may pass along axis 16 over a guidewire support surface 36 of the handle 18 exposed outside of the syringe tube 12 when the piston 22 is drawn back slightly from the distal end of the syringe tube 12. Referring now also to FIG. 2, the guidewire support surface 36 may be, for example, a planar surface extending parallel to the axis 16 by at least ½ inch and preferably by at least 1 inch. Extending upwardly from the guidewire support surface 36 is a series of ridges 40 along which the guidewire 32 may slide. The ridges 40 may be oriented to extend perpendicular to the axis 16 across the full width of the handle 16 to separate the guidewire 32 from the guidewire support surface 36. The upper edges of the ridges 40 may be aligned with the axis 16 minimizing deflection of the guidewire 32.

After passing over the guidewire support surface 36, the guidewire 32 enters into a bore 42 in the handle 18 extending along the axis 16 to be received by a seal chamber 44 in the handle 18.

The seal chamber 44 holds a seal assembly 46 allowing passage of the guidewire 32 therethrough but resisting the passage of liquids before the guidewire 32 has been inserted through the seal 46. Seal 46 may be, for example, a rigid tube 45 having a radially extending flange 47 received in a corresponding groove in the seal chamber 44. Opposite ends of the tube 45 may be covered by thin elastomeric membranes 45 that may be pierced by the guidewire 32 to allow passage of the guidewire 32 therethrough while conforming to the outer surface of the guidewire 32 to minimize leakage.

The seal chamber 44 may be created by assembly together of two portions of the handle 18 through inter-engaging threads 48. In this way the seal chamber 44 may be manufactured by conventional injection molding of two halves of the handle 18 each having portions of the seal chamber 44 separated about the groove receiving the radially extending flange 47, insertion of the seal assembly 46 into one half of the handle 18, and assembly of the threads 48 to close the seal chamber 44 about the seal 46.

After passing through the seal 46, the guidewire 32 extends into a guide tube 50, for example, being a length of stainless steel tubing position coaxially within and insertion-molded into the thermoplastic of the needle connector 21 or otherwise adhered thereto. The guide tube 50 has a central lumen aligned with the axis 16 and may be loosely received in an axial bore in the center of the piston 22 and plunger 14 when the piston 22 is fully inserted to the distal end of the syringe tube 12. The length of this extension of the tube guide 50 into the piston 22 is such as to allow a retraction of the piston 22 for aspiration during the Seldinger or similar techniques (used to ensure proper insertion of the needle) to draw at least 2 ml of fluid while still ensuring that the tube guide 50 fully surrounds the guidewire 32 in its path from the distal end of the piston 22 out of the needle connector 21. It will be generally appreciated that alternatively the guide tube 50 may be fixedly attached to the piston 22 and plunger 14 may extend distally from the piston 22 to be received loosely within an oversized lumen of the needle connector 21.

Referring now also to FIG. 3, during use of the syringe assembly 10, the plunger 14 may be moved with respect to the syringe tube 12 in the manner of a conventional syringe with the physician's index finger 52, thumb 56 and middle finger 54 stabilizing the portion of the plunger 14 extending out of the syringe tube 12. The physician's thumb 56 may then move the plunger 14 by pressing variously on different sides of flange 24 for extension and retraction of the plunger 14 within the syringe tube 12. This manipulation of the plunger 14 can be done with either the index finger 52 or middle finger 54 restraining the syringe tube by finger stops 26. Once the plunger 14 is properly positioned, again without the need to move the thumb 56 or middle finger 54, the guidewire 32 may be retracted or inserted by pressure of the index finger 52 downwardly against the guidewire support surface 36 on which the guidewire 32 is exposed and supported. Motion of the plunger 14 with respect to the syringe tube 12 is resisted by the natural friction between the piston 22 and the syringe to 12. Other methods of operation are also contemplated allowing for a single hand to be used to both operate the syringe for aspiration and to move the guidewire 32. For example, the plunger may be held between the physician's middle finger and thumb and the index finger used to move the guidewire 32. It will be appreciated that the syringe assembly 10 may also be used in a conventional fashion, for example, removing the needle 60 from the syringe tube 12.

Referring now to FIGS. 1 and 4, after insertion of the needle 60 (shown in FIG. 3), the plunger 14 may be drawn backward to draw fluid into the volume of the syringe tube 12 confirming proper needle placement. In this retracted position, the guide tube 50 will desirably be partially inserted into the piston 22 or close to the distal end of the piston 22 to provide positive guidance of the guidewire 32 into the needle connector 21 as it passes through the piston 22. Also in this position, the guidewire support surface 36 will be removed from the syringe tube 12 to be exposed for movement by the physician's finger using the hand holding the syringe assembly 10 as previously discussed with respect to FIG. 3. The guidewire 32 may then be manipulated to pierce the seal assembly 46 and a pass downward through the piston 22 into the guide tube 50 and out through the needle connector 21

The ridges 40 operate primarily to remove contact between the physician's finger 52 (shown in FIG. 4) and the surface of the underlying the ridges 40 minimizing friction between the finger 52 and the plunger 14 such as would move the plunger 14 and concentrating the forces on the guidewire 32 for movement of the guidewire 32. In this respect, the height of the ridges 40 is such as to prevent contact between the finger 52 and the surface beneath the ridges 40, and the tops of the ridges 40 may be upwardly convex (as depicted) to further minimize this contact. In this regard, the material of the plunger 14 may be a slippery polymer such as a polypropylene and may extend upwardly by a distance of greater than 2 mm and preferably greater than 4 mm and may be separated by a distance of at least 5 mm along the axis 16.

Referring now to FIG. 5, in an alternative embodiment, the guidewire 32 may be advanced by means of a intermediate element 62, in this case, a flexible pawl attached to the plunger handle 18 by living hinges 64 to be pressed down, for example, by the index finger 52 and to extend as shown by dotted lines against the guidewire 32 to move the guidewire forward along the guidewire support surface 36. Release of the pawl decreases the frictional force between a distal end of the pawl and the guidewire 32 allowing the pawl to retract without bringing the guidewire 32 along with it. Such a design also permits use of a high frictional material on the distal end of the pawl such as can increase its gripping power over that which might be available from a gloved finger. Other similar methods of indirect advancement of the guidewire 32 may, for example, include intervening wheels having frictional surfaces contacting one or both of the guidewire 32 and the finger 52. More generally a ratcheting system may be employed in which a single direction of movement of the guidewire 32 is enforced by guides, and the user's finger or an intervening element serve to press down on a compliant support element deforming the guidewire 32 in the region of the guidewire support surface 36. This pressing down straightens the guidewire 32 progressing it along the guides.

When such an intervening element is added, the need for the guidewire support surface 36 to be retracted from the syringe tube 12 can be relaxed. In addition, it will be appreciated that an extension of the length of the handle 18 can allow the support surface to be exposed with minimal or no retraction of the plunger 14.

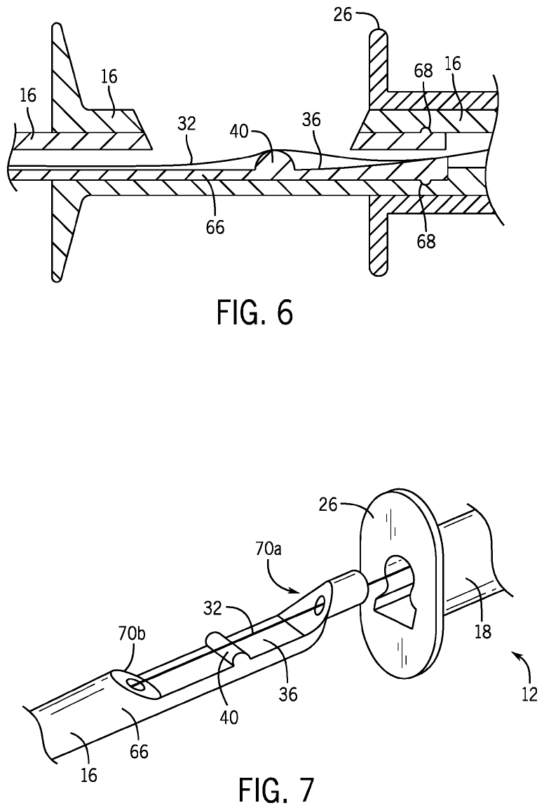

Referring now to FIGS. 6 and 7, in one embodiment, the handle 16 may include a removable central axial insert 66 connecting with remaining parts of the handle 16, for example, by a detent or catch 68 to operate as an integrated handle as described above with respect to FIGS. 1 and 2. The insert 66 may include the guidewire support surface 36 and a ridge 40, both separating apertures 70a and 70b along the path of the guidewire 32. The removable insert 66 permits the guidewire 32 to be pre-installed through retaining apertures 70a and 70b on the removable insert 66, for example, the latter as may be attached to the exit trough 30 of the guidewire dispenser 28. This eliminates the need to thread the flexible guidewire 32 into the handle 18 as fully assembled at the time of the procedure. Instead the threading is effectively performed by inserting the removable insert 66, already holding the guidewire 32, into the handle 16, moving this time-consuming step to the earlier time of installation of the guidewire 32 in the guidewire dispenser 32 and away from the immediacy of the insertion of the guidewire in the blood vessel.

As used herein, the term “finger” shall be considered to include the thumb.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom”, and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including”, and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A syringe assembly comprising:
a syringe tube having a needle connector at a first end in fluid communication with a volume of the syringe tube; and
a syringe plunger providing a piston slidably received within the syringe tube and a handle portion extending from a second end of the syringe tube;
wherein the syringe plunger includes a bore passing through the piston allowing a passage of a guidewire received within the second end of the syringe tube to extend through the piston to the needle connector; and
wherein the handle portion provides a guidewire support surface against which the guidewire may be pressed to advance or retract the guidewire through the bore.

2. The syringe assembly of claim 1 wherein the guidewire surface is exposable with movement of the syringe plunger outside of the syringe tube so that the guidewire may be pressed by a user's finger to advance or retract the guidewire through the bore.

3. The syringe assembly of claim 1 wherein the guidewire support surface includes at least one ridge extending away from the handle and extending across a length of the guidewire when the latter is positioned within the syringe assembly.

4. The syringe assembly of claim 1 further including a guidewire holder attached to the handle portion for dispensing a guidewire from the guidewire holder to the handle.

5. The syringe assembly of claim 4 wherein the guidewire holder attaches to an end of the handle portion removed from the piston and provides a slot extending from the guidewire holder to the guidewire support system and opens to allow insertion of a guidewire therethrough in a direction perpendicular to a direction of advancing or retracting the guidewire through the bore.

6. The syringe assembly of claim 1 wherein the needle connector provides a central lumen along an axis and wherein the bore is aligned with the axis and movement of the syringe plunger is along the axis.

7. The syringe assembly of claim 1 further including a guide tube extending from the needle connector toward the syringe plunger and wherein the syringe plunger provides an axial plunger bore and wherein the guide tube is sized to be received within the axial plunger bore for at least some positions of the syringe plunger within the syringe tube.

8. The syringe assembly of claim 1 wherein a seal is positioned within the bore to restrict the passage of liquids through the bore.

9. The syringe assembly of claim 1 wherein the guidewire support surface is adapted to be proximate to a finger of a user's hand, when that hand is supporting the syringe plunger, to advance the guidewire with the finger.

10. A method of inserting a guidewire in a vessel using a syringe assembly having:
a syringe tube having a needle connector at a first end in fluid communication with a volume of the syringe tube; and
a syringe plunger providing a piston slidably received within the syringe tube and a handle portion extending from a second end of the syringe tube;
wherein the syringe plunger includes a bore passing through the piston allowing a passage of a guidewire received within the second end of the syringe tube to extend through the piston to the needle connector; and
wherein the handle portion provides a guidewire support surface against which the guidewire may be pressed to advance or retract the guidewire through the bore; the method comprising
(a) inserting a needle attached to the needle connector into the vessel;
(b) supporting the syringe tube with a hand while using a finger of the hand to advance or retract the guidewire along the support surface through the bore.
